(19) United States
(12) Patent Application Publication
(10) Pub. No.: US 2019/0351006 A1
(43) Pub. Date: Nov. 21, 2019

(54) METHODS OF ADMINISTRATION OF NMDA RECEPTOR AGONISTS

(71) Applicant: Naurex Inc., Madison, NJ (US)

(72) Inventors: Pradeep Banerjee, Hillsborough, NJ (US); Herbert Meltzer, Evanston, IL (US)

(21) Appl. No.: 16/185,522

(22) Filed: Nov. 9, 2018

Related U.S. Application Data
(60) Provisional application No. 62/584,652, filed on Nov. 10, 2017.

(51) Int. Cl. A61K 38/07

(52) U.S. Cl. CPC A61K 38/07

(57) ABSTRACT

Disclosed are methods for treating a cognitive or neurological diseases and disorders comprising the step of administering rapastinel or a pharmaceutically acceptable salt, ester or metabolite thereof to a patient in need thereof, wherein rapastinel is administered for an administration period of about one to about fourteen days followed by a holiday period of at least about one to sixteen weeks wherein rapastinel is not administered.

### BACKGROUND

An N-methyl-D-aspartate (NMDA) receptor is a postsynaptic, ionotropic receptor that is responsive to, inter alia, the excitatory amino acids glutamate and glycine and the synthetic compound NMDA. The NMDA receptor (NMDAR) appears to controls the flow of both divalent and monovalent ions into the postsynaptic neural cell through a receptor associated channel and has drawn particular interest since it appears to be involved in a broad spectrum of CNS disorders. The NMDAR has been implicated, for example, in neurodegenerative disorders including stroke-related brain cell death, convulsive disorders, and learning and memory. NMDAR also plays a central role in modulating normal synaptic transmission, synaptic plasticity, and excitotoxicity in the central nervous system. The NMDAR is further involved in Long-Term Potentiation (LTP), which is the persistent strengthening of neuronal connections that underlie learning and memory The NMDAR has been associated with other disorders ranging from hypoglycemia and cardiac arrest to epilepsy. In addition, there are preliminary reports indicating involvement of NMDA receptors in the chronic neurodegeneration of Huntington's, Parkinson's, and Alzheimer's diseases. Activation of the NMDA receptor has been shown to be responsible for post-stroke convulsions, and, in certain models of epilepsy, activation of the NMDA receptor has been shown to be necessary for the generation of seizures. In addition, certain properties of NMDA receptors suggest that they may be involved in the information-processing in the brain that underlies consciousness itself. Further, NMDA receptors have also been implicated in certain types of spatial learning.

In view of the association of NMDAR with various disorders and diseases, NMDA-modulating small molecule agonist and antagonist compounds have been developed for therapeutic use. NMDA receptor compounds may exert dual (agonist/antagonist) effect on the NMDA receptor through the allosteric sites. These compounds are typically termed “partial agonists”. In the presence of the principal site ligand, a partial agonist will displace some of the ligand and thus decrease Ca⁺⁺ flow through the receptor. In the absence of the principal site ligand or in the presence of a lowered level of the principal site ligand, the partial agonist acts to increase Ca⁺⁺ flow through the receptor channel.

Recently, a partial agonist of NMDAR with the following structure has been reported (rapastinel or GLYX-13):

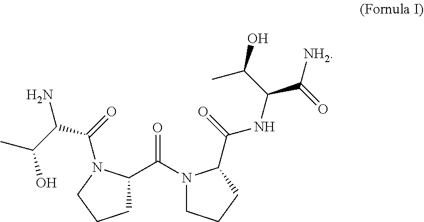

PCT/US2017/015851 describes a process for synthesis of peptide compounds, including rapastinel, the contents of which are incorporated herein by reference in its entirety.

### SUMMARY

Provided herein are methods for treating, preventing or improving neurological or cognitive functions in a patient in need thereof, the method comprising administering to the patient a therapeutically effective amount rapastinel or a pharmaceutically acceptable salt, ester or metabolite thereof for a period of Interval A, wherein the administration provides therapeutic effect for a period of two, three, four, five, six, seven, eight, nine, ten, eleven or twelve times of Interval A.
Provided herein are methods for methods for administering a compound of Formula I, or a pharmaceutically acceptable salt, ester or metabolite thereof to a patient in need thereof wherein said compound is administered for a period of one, two, three, four, five, six, seven, eight, nine or ten days; wherein therapeutic effect of said compound last for one, two, three, four, five, six, seven, eight, nine, ten, eleven, twelve, thirteen, thirteen or or more days.
In some embodiments, provided are methods for treating or preventing a disease or disorder selected from the group consisting of deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression; attention-deficit/hyperactivity disorder; schizophrenia including negative symptoms; schizophreniform disorder; schizoaffective disorder including of the delusional type or the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy; Parkinson's disease: restless leg syndrome; disorders comprising as a symptom thereof a deficiency in cognition. The methods and regimens disclosed herein include administering a particular dose (or a range of doses) of rapastinel (or a composition containing rapastinel) at a particular frequency (or a range of frequencies, e.g., b.i.d for one, two, three, four, five, six or seven days) over a time period that is sufficient so as to provide the patient with a prolonged therapeutic effect (e.g., two, three, four, five, six, seven, eight, nine, ten, eleven, twelve weeks) over said time period. For example, the administration of rapastinel, or a pharmaceutically acceptable salt, ester or metabolite thereof for about a week (once, twice or three times a day) provides therapeutic effect for a period of about two, three, four, five, six, seven, eight, nine, ten, eleven, twelve, thirteen or fourteen weeks. The frequency of administration allows for a period of administration followed a period of drug-holiday period.

### BRIEF DESCRIPTION OF DRAWINGS

FIG. 1 Rapastinel transiently restores NOR in scPCP mice in an mTOR signaling pathway-dependent manner. The scPCP-treated mice (B) showed reduced DI vs vehicle-treated mice (A) (***p<0.001); rapastinel (1 mg/kg; subcu) given 30 min before AT rescued NOR deficit in scPCP-treated mice (###p<0.001; (C). Pretreatment with rapamycin (0.2 nM; ICV) 24 h prior to rapastinel (1 mg/kg; subcu) significantly blocked the efficacy of rapastinel (+++p<0.001; D).

FIG. 2 Subchronic rapastinel (3 days; b.i.d) produced prolonged restoration of NOR in subchronic PCP mice for 9 weeks post injections. A single dose of rapastinel 1 mg/kg restored NOR in week 10 (not further tested). The subchronic PCP-treated mice (B) showed reduced DI vs veh-treated mice (A) (***p<0.001); rapastinel (1 mg/kg; sc) given for 3 days; bid; rescued NOR deficit in scPCPtreated mice for up to 9 weeks (###p<0.001; C-L). Acute treatment with a single dose of rapastinel (1 mg/kg; sc; N) on week 11, 30 min prior to AT, significantly rescued the reduction in DI in week 10.

FIG. 3. The subchronic PCP-treated mice (B) showed reduced DI vs veh-treated mice (A) (***p<0.001); rapastinel (1 mg/kg; sc) given for 5 days; bid; rescued NOR deficit in scPCP-treated mice for at least 10 weeks (###p<0.001; C-M).

FIG. 4 Rapastinel transiently restores reversal learning in subchronic PCP mice in an mTORsignaling pathway-dependent manner The subchronic PCP-treated mice (B) showed reduced percent correct responses vs veh-treated mice (A) (***p<0.001); rapastinel (1 mg/kg; sc) given 30 min before reversal phase of the RL task, rescued RL deficit in scPCP-treated mice (###p<0.001; C). Pretreatment with rapamycin (0.2 nM; ICV) 24 h prior to rapastinel (3 mg/kg; sc) significantly blocked the efficacy of rapastinel (+++p<0.001; D).

FIG. 5. Rapastinel (1 mg/kg, subcu) produced small selective increase in DA efflux in mPFC, but not in HIP. Ketamine produced marked increases in ACh, DA, 5-HT, NE, DOPAC, HVA, 5-HIAA, and Glu, in both mPFC and HIP [FIGS. 5a,b,c]. Rapastinel increased mPFC DA efflux (*p<0.05), while ketamine produced very large increases in the efflux of ACh, DA, 5-HT, DOPAC, 5-HIAA, and glutamate, and serine. Ketamine, but not rapastinel, produced similar large increases in hippocampal NT efflux. FIG. 5c. Baseline DA following vehicle, rapastinel (1 mg/kg), ketamine (30 mg/kg), and PCP (10 mg/kg) in mPFC.

FIG. 6. FIG. 6a. Baseline DA in mPFC in Saline, scPCP, and scPCP+scRAP groups; *p<0.05: decrease in DA vs saline. FIG. 6(b,c): Activity dependent changes in DA, Glu, and ACh in mPFC and HIP in control, scPCPtreated mice with and without post-PCP rapastinel during NOR Acquisition (A) and retrieval (R).

FIG. 7. Drug treatment protocol diagram.

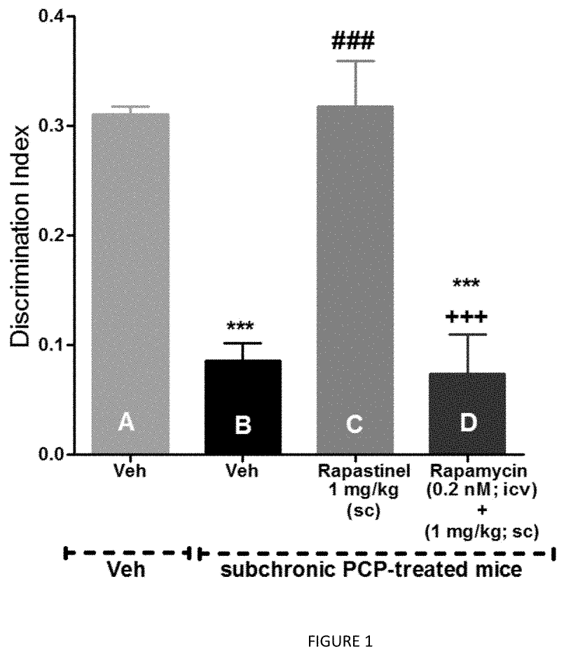

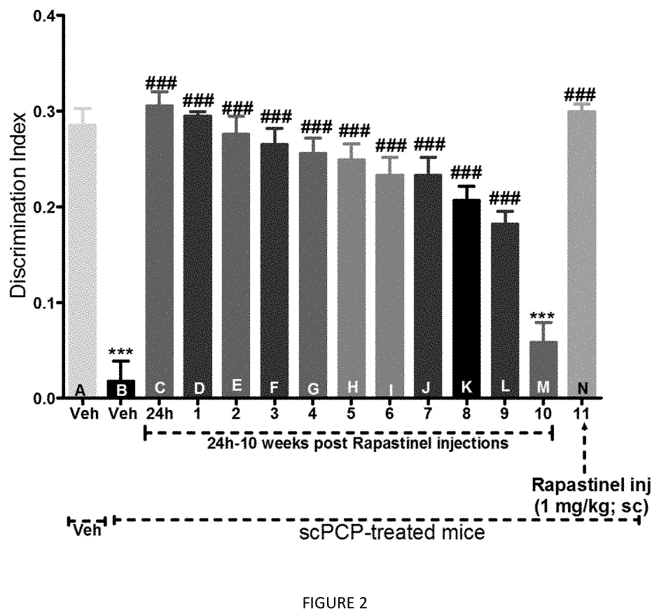

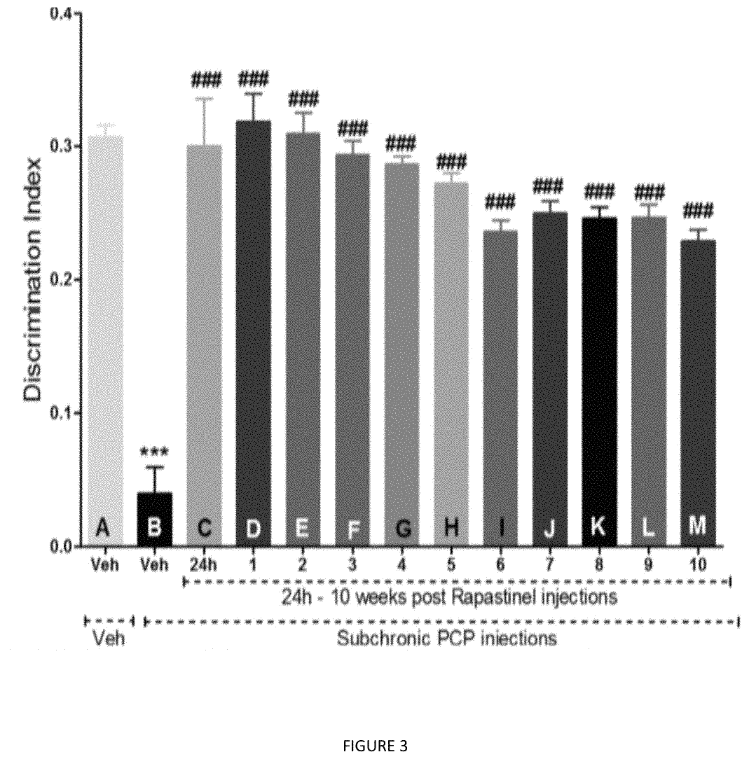

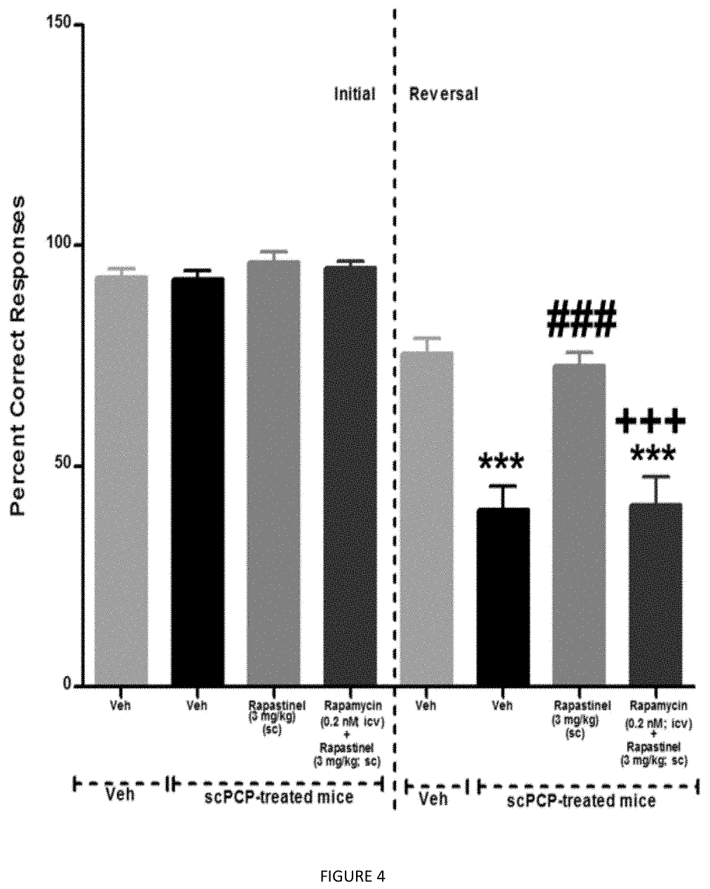

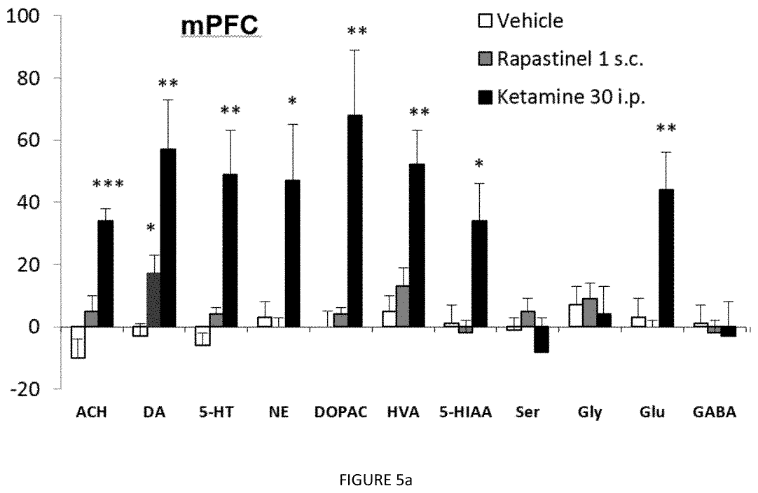

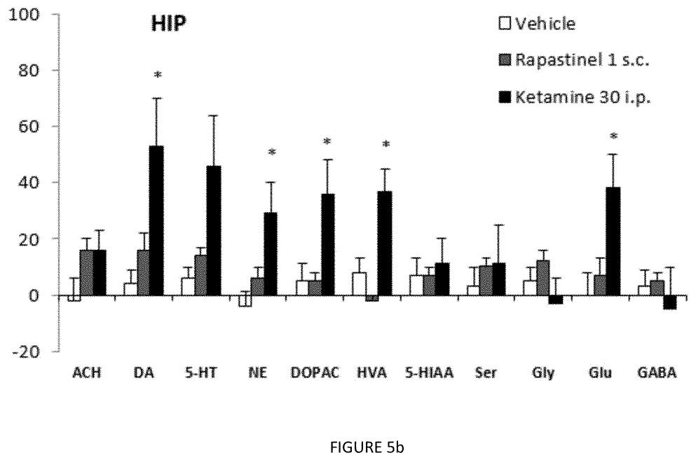

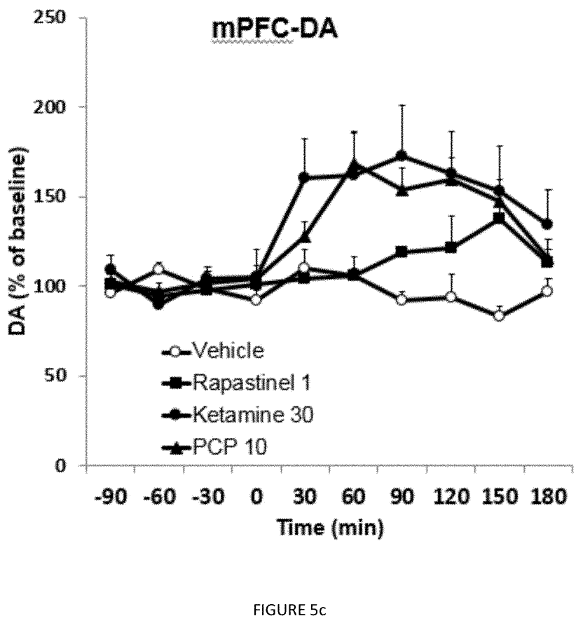

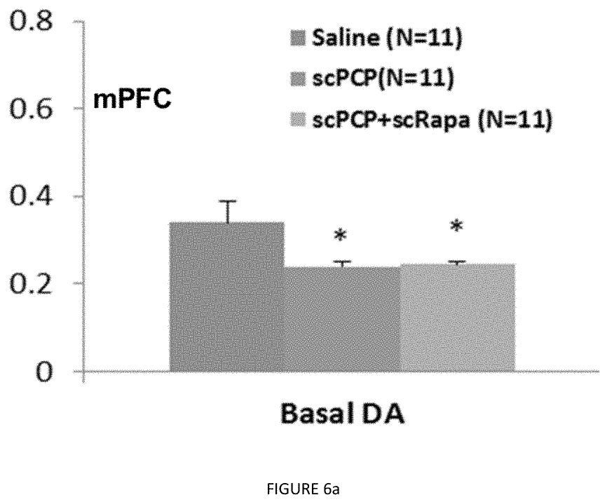

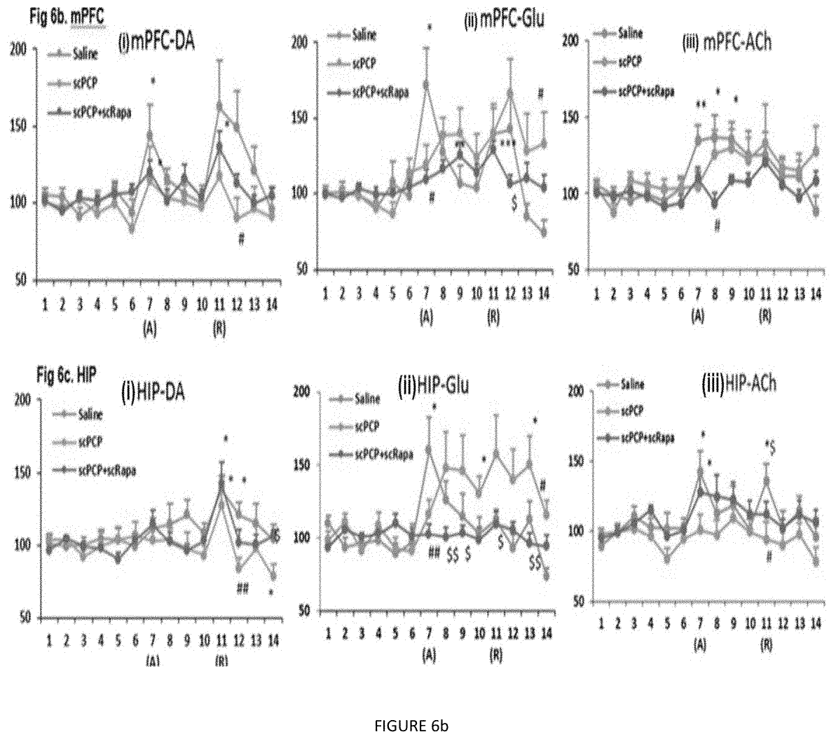

### DETAILED DESCRIPTION

Provided herein are methods for treating, preventing or improving neurological or cognitive functions in a patient in need thereof, the method comprising administering to the patient a therapeutically effective amount rapastinel or a pharmaceutically acceptable salt, ester or metabolite thereof for an administration period, wherein the administration provides therapeutic effect for a period of two, three, four, five, six, seven, eight, nine, ten, eleven or twelve times of the administration period.

Provided herein are methods for methods for administering a compound of Formula I, or a pharmaceutically acceptable salt, ester or metabolite thereof to a patient in need thereof wherein said compound is administered for a period of one, two, three, four, five, six, seven, eight, nine or ten days; wherein therapeutic effect of said compound last for one, two, three, four, five, six, seven, eight, nine, ten, eleven, twelve, thirteen, thirteen or or more days.

In some embodiments, provided are methods for treating or preventing a disease or disorder selected from the group consisting of deficiency in memory, intellect, or learning and logic ability; reduction in any particular individual's functioning in one or more cognitive aspects; age-related cognitive decline; dementia; Alzheimer's disease; multi-infarct dementia; alcoholic dementia or other drug-related dementia; dementia associated with intracranial tumors or cerebral trauma; dementia associated with Huntington's disease or Parkinson's disease; AIDS-related dementia; delirium; amnestic disorder; mental retardation; a learning disorder including reading disorder, mathematics disorder, or a disorder of written expression; attention-deficit/hyperactivity disorder; schizophrenia including negative symptoms; schizophreniform disorder; schizoaffective disorder including of the delusional type or the depressive type; delusional disorder; substance-induced psychotic disorder; personality disorder of the paranoid type; personality disorder of the schizoid type; panic disorder; phobias; obsessive-compulsive disorder; stress disorders; generalized anxiety disorder; movement disorders involving Huntington's disease; dyskinesia associated with dopamine agonist therapy; Parkinson's disease: restless leg syndrome; disorders comprising as a symptom thereof a deficiency in cognition. The methods and regimens disclosed herein include administering a particular dose (or a range of doses) of rapastinel (or a composition containing rapastinel) at a particular frequency (or a range of frequencies, e.g., b.i.d for one, two, three, four, five, six or seven days) over a time period that is sufficient so as to provide the patient with a prolonged therapeutic effect (e.g., two, three, four, five, six, seven, eight, nine, ten, eleven, twelve weeks) over said time period. For example, the administration of rapastinel, or a pharmaceutically acceptable salt, ester or metabolite thereof for about a week (once, twice or three times a day) provides therapeutic effect for a period of about two, three, four, five, six, seven, eight, nine, ten, eleven, twelve, thirteen or fourteen weeks. The frequency of administration allows for a period of administration followed a period of drug-holiday period. The period of time during which the patient receives the two or more doses is sometimes referred to herein as an “induction period of time” (also sometimes referred to herein as “repeat” or “repeated” dosing). The methods and regimens described herein can further include a “rest period of time,” during which time the patient does not receive rapastinel (or a composition containing the same). In some embodiments, the methods and regimens include two or more treatment cycles (e.g. continuous cycles), in which each cycle includes an induction period of time and a rest period of time. As the skilled person will appreciate, each of the treatment cycles can be independently varied from one another in terms of dosage, frequency, duration of induction period of time, duration of rest period of time, etc.

“Treating” includes any effect, e.g., lessening, reducing, modulating, or eliminating, that results in the improvement of the condition, disease, disorder and the like. “Individual,” “patient,” or “subject” are used interchangeably and include any animal, including mammals, preferably mice, rats, other rodents, rabbits, dogs, cats, swine, cattle, sheep, horses, or primates, and most preferably humans.

The term “effective amount” refers to an amount of the subject component, e.g., GLYX-13 (or a composition containing GLYX-13) that will elicit the biological or medical response of a tissue, system, animal or human that is being sought by the researcher, veterinarian, medical doctor or other clinician.

GLYX-13 may be obtained by recombinant or synthetic methods such as those described in U.S. Pat. Nos. 5,763,393 and 4,086,196 herein incorporated by reference. Also contemplated are polymorphs, hydrates, homologs, solvates, free bases, and/or suitable salt forms of GLYX 13 such as, but not limited to, the acetate salt. The peptide may be in cyclized or non-cyclized form as further described in U.S. Pat. No. 5,763,393. In some embodiments, a GLYX-13 analog may include an insertion or deletion of a moiety on one or more of the Thr or Pro groups such as a deletion of CH₂, OH, or NH₂ moiety. In other embodiments, GLYX-13 may be optionally substituted with one or more halogens, C₁-C₃ alkyl (optionally substituted with halogen or amino), hydroxyl, and/or amino. Other compounds contemplated for use herein include Glycine-site partial agonists of the NMDAR disclosed in U.S. Pat. Nos. 5,763,393, 6,107,271, and Wood et al., Neuro. Report, 19, 1059-1061, 2008, the entire contents of which are herein incorporated by reference.

In some embodiments, a therapeutically effective amount of GLYX-13 for adult human treatment administered, for example, during an induction period of time, are in the range of about 0.01 mg/kg to about 1000 mg/kg per administration (e.g., about 0.01 mg/kg to about 100 mg/kg, about 0.01 mg/kg to about 50 mg/kg, about 0.01 mg/kg to about 25 mg/kg, about 0.01 mg/kg to about 10 mg/kg, about 0.1 mg/kg to about 100 mg/kg, about 0.1 mg/kg to about 50 mg/kg, about 0.1 mg/kg to about 50 mg/kg, about 0.1 mg/kg to about 10 mg/kg, about 1 mg/kg to about 100 mg/kg, about 1 mg/kg to about 50 mg/kg, about 1 mg/kg to about 50 mg/kg per day, about 1 mg/kg to about 10 mg/kg, or about 1 mg/kg to about 10 mg/kg per administration, e.g., once a week, twice a week or three times a week and/or as described herein). The dosage of GLYX-13 may be at any dosage including, but not limited to, about 1 ug/kg, 25 ug/kg, 50 ug/kg, 75 ug/kg, 100 u ug/kg, 125 ug/kg, 150 ug/kg, 175 ug/kg, 200 ug/kg, 225 ug/kg, 250 ug/kg, 275 ug/kg, 300 ug/kg, 325 ug/kg, 350 ug/kg, 375 ug/kg, 400 ug/kg, 425 ug/kg, 450 ug/kg, 475 ug/kg, 500 ug/kg, 525 ug/kg, 550 ug/kg, 575 ug/kg, 600 ug/kg, 625 ug/kg, 650 ug/kg, 675 ug/kg, 700 ug/kg, 725 ug/kg, 750 ug/kg, 775 ug/kg, 800 ug/kg, 825 ug/kg, 850 ug/kg, 875 ug/kg, 900 ug/kg, 925 ug/kg, 950 ug/kg, 975 ug/kg, 1 mg/kg, 2.5 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg. In certain embodiments, GLYX-13 may be therapeutically effective for depression with a range (e.g., an intravenous dose range) of about 1 to about 10 mg/kg, e.g., about 5 to about 10 mg/kg, e.g. about 1 mg/kg, about 5 mg/kg, or about 10 mg/kg.

In some embodiments, a therapeutically effective amount of GLYX-13 for adult human treatment administered, for example, during an induction period (administration period) of time may be a fixed dose of about 1000 mg to about 200 mg, or 900 mg to about 100 mg e.g., about 200 mg to about 500 mg, e.g., 50 mg, 100 mg, 225 mg, 250 mg, 200 mg, 300 mg, 350 mg, 450 mg, 500 mg, 600 mg, 700 mg, 750 mg, and/or 900 mg unit dose. It will be appreciated that a maintenance dose may be lower than the induction dose.

In some embodiments, any of the GLYX-13 dosages described herein can be administered on a less than daily basis, e.g., every other day (e.g., every two days); one or two times a week; one, two or three times a week; two or three times a week; twice weekly (e.g. every 3 days, every 4 days, every 5 days, every 6 days or e.g. administered with an interval of about 2 to about 3 days between doses); every three to four days; once a week; once every two weeks (bi-weekly); twice monthly; once a month or even less often. In certain embodiments, GLYX-13 is administered at a frequency of once a week, twice a week, once every two weeks, or any combination thereof.

In certain embodiments GLYX-13 (rapastinel) is administered at a range (e.g., an intravenous dose range) of about 1 to about 10 mg/kg, e.g., about 5 to about 10 mg/kg, e.g. about 1 mg/kg, about 5 mg/kg, or about 10 mg/kg, and/or GLYX-13 is administered at a frequency of once a week, once every two weeks, or any combination thereof.

In some embodiments, the methods and regimens include two or more treatment cycles (e.g. continuous cycles), in which each cycle includes an induction period of time and a rest period of time. As the skilled person will appreciate, each of the treatment cycles can be independently varied from one another in terms of dosage, frequency, duration of induction period of time, duration of rest period of time, etc.

### Administration and Formulations

GLYX-13 as well as any other pharmacological agent (e.g., one or more other antidepressant agents) of the present invention may be administered by various means, depending on their intended use, as is well known in the art. For example, if compositions of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations, or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In some embodiments, GLYX-13 herein may be administered parenterally to a patient including, but not limited to, subcutaneously, intramuscularly, and intravenously. In some embodiments, one or more of the components of the combinations described herein may also be administered via slow controlled i.v. infusion or by release from an implant device.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, intranasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards. The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The combinations described herein may also contain other active compounds providing supplemental, additional, or enhanced therapeutic functions. Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

Disclosed compounds may be provided as part of a liquid or solid formulation, for example, aqueous or oily suspensions, solutions, emulsions, syrups, and/or elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl hydroxybenzoate and sorbic acid. Contemplated compounds may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The present invention has multiple aspects, illustrated by the following non-limiting examples.

### Examples

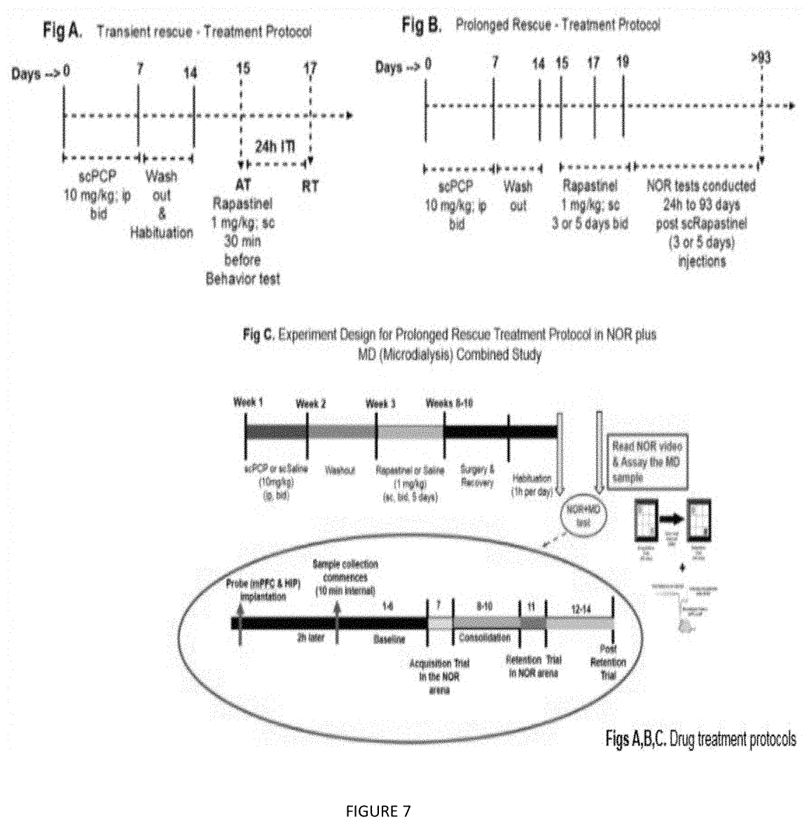

Animals: Male C57BL/6J mice 22-25 g (21/2 to 3 month old) were used is these studies. Drugs: Rapastinel was obtained from SAI Life Sciences (India). Rapastinel, PCP, and ketamine were dissolved in 0.9% sterile saline (Sal). PCP and ketamine were administered intraperitoneally (ip), at a volume of 10 mL/kg body weight. Rapastinel was given subcutaneously (subcu), and rapamycin was given intracerebroventricularly (icy). Drug Treatments: Male C57BL/6J mice were randomly assigned to treatment groups of 10 per group. To develop the cognitive deficit, groups of 10 mice were treated with PCP (10 mg/kg i.p., twice a day for 7 days) followed by a 7-day washout period. Following the washout period, the subchronic PCP mice were tested to determine that an NOR deficit had been established. See Rajagopal et al 3,4. See FIG. 7, for treatment schedules.
Behavior, in vivo Microdialysis (MD) and ICV Procedures—The procedures have been described in detail in References 3,10,11,12,13. See FIG. 7. for pictorial representations of drug treatment protocols.
Rapastinel's ability to restore episodic memory (NOR) and cognitive flexibility (RL), like its antidepressant action in rodents, is mTOR dependent as indicated by blockade by ICV rapamycin. Subchronic rapastinel (1 mg/kg, 3 days, bid, subcu) produced prolonged reversal of scPCP-induced NOR deficit for up to 9 weeks suggesting that repeated treatments with rapastinel, at the appropriate schedule, has potential as a cognitive enhancer in schizophrenia and other conditions, e.g. aging.
Subchronic rapastinel (1 mg/kg, 5 days, bid, subcu) showed prolonged reversal of scPCP-induced NOR deficit longer compared to 3 day treatment.
Increase in cortical DA efflux, in transient and subchronic rescue by rapastinel, may be important for triggering neuroplastic changes in AMPAR modulation of the activity of principal neurons in mPFC and HIP.

### REFERENCES

1) Moskal et al (2017). Curr Neuropharmacol 15(1): 47-56; 2) Moska et al (2005). Neuropharmacology 49(7): 1077-1087;
3) Rajagopal et al (2016). Behav Brain Res 299: 105-110.
4) Burgdorf et al (2015). Behav Brain Res 294: 177-185.
5) Lu et al (2014). Int J Neuropsychopharmacol 18(5).
6) Liu et al (2017). Neuropsychopharmacology 42(6): 1231-1242.
7) Horiguchi et al (2012). Neuropsychopharmacology 37(10): 2175-2183.
8) Horiguchi et al. (2016). Behav Brain Res 301: 132-141.
9) Oyamada et al (2015). Behav Brain Res 285: 165-175.
10) Rajagopal et al (2017). Behav Brain Res 332: 180-199.
11) Rajagopal et al. (2016b). Psychopharmacology (Berl) 233(4): 649-660.
12) Huang et al. (2014). J Neurochem 128(6): 938-949.
13) Zhou et al (2013). Neuron 77(4): 647-654

1. A method for treating a cognitive or neurological disease, defect or disorder comprising the step of administering rapastinel or a pharmaceutically acceptable salt, ester or metabolite thereof to a patient in need thereof, wherein rapastinel is administered for an administration period of about one to about fourteen days followed by a holiday period of at least about one to sixteen weeks wherein rapastinel is not administered.

2. The method according to claim 1 wherein said administration period is about two days.

3. The method according to claim 1 wherein said administration period is about three days.

4. The method according to claim 1 wherein said administration period is about four days.

5. The method according to claim 1 wherein said administration period is about five days.

6. The method according to claim 1 wherein said administration period is about six days.

7. The method according to claim 1 wherein said administration period is about seven days.

8. The method according to claim 1 wherein said administration period is about eight days.

9. The method according to claim 1 wherein said administration period is about nine days.

10. The method according to claim 1 wherein said administration period is about ten days.

11. The method according to claim 1 wherein said administration period is about eleven days.

12. The method according to claim 1 wherein said administration period is about twelve days.

13-32. (canceled)

33. The method according to any of claim 1 wherein said cognitive disease or disorder is selected from:
deficiency in memory, intellect, learning, logic ability;
reduction in any particular individual's functioning in one or more cognitive aspects;
age-related cognitive decline;
dementia;
Alzheimer's disease;
multi-infarct dementia;
alcoholic dementia or other drug-related dementia;
dementia associated with intracranial tumors or cerebral trauma;
dementia associated with Huntington's disease or Parkinson's disease;
AIDS-related dementia;
delirium; amnestic disorder;
mental retardation;
a learning disorder including reading disorder, mathematics disorder, or a disorder of written expression;
attention-deficit/hyperactivity disorder;
schizophrenia including negative symptoms;
schizophreniform disorder;
schizoaffective disorder including of the delusional type or the depressive type;
delusional disorder;
substance-induced psychotic disorder; personality disorder of the paranoid type; personality disorder of the schizoid type;
panic disorder; phobias;
obsessive-compulsive disorder;
stress disorders;
generalized anxiety disorder;
movement disorders involving Huntington's disease;
dyskinesia associated with dopamine agonist therapy;
Parkinson's disease:
restless leg syndrome;
disorders comprising as a symptom thereof a deficiency in cognition.
